(19) United States
(12) Patent Application Publication
(10) Pub. No.: US 2025/0339410 A1
(43) Pub. Date: Nov. 6, 2025

(54) TREATMENT FOR AUTISM SPECTRUM DISORDER

(71) Applicant: STALICLA SA, Geneva (CH)

(72) Inventors: Lynn Durham, Geneva (CH); Jean-Marc Hyvelin, Geneva (CH)

(21) Appl. No.: 19/086,529

(22) Filed: Mar. 21, 2025

(30) Foreign Application Priority Data
Mar. 21, 2024 (CH) 000304/2024

(51) Int. Cl. A61K 31/437; A61K 31/196; A61P 25/00

(52) U.S. Cl. CPC A61K 31/437; A61K 31/196; A61P 25/00

(57) ABSTRACT

The invention relates to pharmaceutical composition comprising ibudilast and bumetanide as active ingredients for use in the treatment of autism spectrum disorder (ASD), wherein the treatment induces favorable changes in the EEG of the patient.

### CROSS-REFERENCE TO RELATED APPLICATIONS

This application claims the benefit of Switzerland Application No. 000304/2025, filed Mar. 21, 2024.

### FIELD OF THE INVENTION

The invention relates to pharmaceutical compositions, kits, and methods for treating autism spectrum disorder.

### BACKGROUND

Autism Spectrum Disorder (ASD) comprises a group of lifelong neurodevelopmental conditions that, according to recent estimates, affect 1.5% of the population in developed countries. According to the current diagnostic criteria DSM-5 (Diagnostic and Statistical Manual of Mental Disorders, 5th Edition), an individual with ASD must show deficits in social interaction and communication combined with at least two of four subdomains of restricted or repetitive behaviors. The manifestations of ASD must cause clinically significant impairment, affecting the ability of patients to interact with others, especially same aged peers in youth.

The current version of the DSM-5 abandoned classifications of subtypes of ASD to group them under one umbrella, mostly due to the lack of objective criteria to define previous subtypes including Asperger's Disorder and Pervasive Developmental Disorder Not Otherwise Specified (PDD-NOS). However, ASD remains characterized by a high heterogeneity in its behavioral manifestations with very complex genetic underpinnings, suggesting the existence of subtypes of ASD. Therefore, efforts to categorize ASD are still of critical importance and must rely on defining a relationship between clinical symptoms and biological mechanisms to improve the outcome of clinical trials.

Previously, a first biologically defined subgroup of patients with ASD, ASD Phenotype 1 (ASD-Phen1), was identified, patients being characterized by the presence of specific, non-behavioral, clinical signs and symptoms (CSSs) mirroring the effects of an over-activation of NRF2 gene and its related pathways (WO 2019/086722 A1). STP1, a combination of ibudilast and bumetanide, was then identified as a potential tailored treatment for ASD-Phen1 (EP 3785733 B1).

Ibudilast is a brain penetrant phosphodiesterase 3 (PDE3), 4 (PDE4), 10 (PDE10), and 11 (PDE11) inhibitor. Bumetanide is an inhibitor of NKCC1/NKCC2. At the physiological level, the increased expression and activation of NKCC1 may lead to the accumulation of chlorine in cells and antagonize the gamma-aminobutyric acid (GABA) current in neurons, thereby decreasing inhibition and favoring excitation. This increased neuronal excitation can then lead to an aberrant neuronal network activity and affect patient behavior. To compensate for this indirect effect of ibudilast, we proposed to combine ibudilast with bumetanide, an inhibitor of NKCC1, for the treatment of ASD patients.

However, the actual effects of STP1 on ASD patients have not been well described yet.

### Objective Technical Problem

The objective technical problem is therefore the provision on a treatment for ASD that shows measurable physiological effects in the patients.

### SUMMARY OF THE INVENTION

In one aspect, the invention relates to a pharmaceutical composition comprising ibudilast and bumetanide as active ingredients for use in the treatment of autism spectrum disorder (ASD), wherein the treatment induces favorable changes in the EEG of the patient.

In another aspect, the invention relates to a kit comprising a dosage form comprising ibudilast and a dosage form comprising bumetanide for use in the treatment of autism spectrum disorder (ASD), wherein the treatment induces at least one favorable change in the EEG of the patient.

In yet another aspect, the invention relates to a method of treating autism spectrum disorder (ASD), wherein the treatment induces favorable changes in the EEG of the patient.

### BRIEF DESCRIPTION OF THE FIGURES

FIGS. 1a-1b show the effect of STP1 on gamma waves. FIG. 1a) Gamma 2 absolute power after administration of STP1 5/1 mg, STP1 10/1 mg and placebo BID in ASD-Phen1 on Day 14; FIG. 1b) Gamma 1 Power (dB) in Frontal and Entorhinal brain regions on Day 14 in placebo (N=3), STP1 5/1 mg (N=6) and STP1 10/1 mg (N=3). Each value is normalized to pre-dose.

FIG. 2 shows that the complex synchronization analysis showing an increased ITC signal indicates increased neural synchronization to the auditory chirp measured in placebo N=2, and STP1 10/1 mg N=3 at Day 1 post-dose and Day 14.

FIGS. 3a-3b shows the effect of STP1 on alpha waves. FIG. 3a) Alpha 1 and Alpha 2 absolute power after administration of STP1 5/1 mg, STP1 10/1 mg and placebo BID in ASD-Phen1 on Day 14; FIG. 3b) Alpha 2 (relative) in brain regions on Day 14 in placebo (N=3), STP1 5/1 mg (N=6) and STP1 10/1 mg (N=3).

### DETAILED DESCRIPTION

In one aspect, the invention relates to a pharmaceutical composition comprising ibudilast and bumetanide as active ingredients for use in the treatment of autism spectrum disorder (ASD), wherein the treatment induces favorable changes in the EEG of the patient.

In another aspect, the invention relates to a kit comprising a dosage form comprising ibudilast and a dosage form comprising bumetanide for use in the treatment of autism spectrum disorder (ASD), wherein the treatment induces favorable changes in the EEG of the patient.

In yet another aspect, the invention relates to a method of treating autism spectrum disorder (ASD), wherein the treatment induces favorable changes in the EEG of the patient. In a preferred embodiment, the treatment comprises administration of a pharmaceutical composition comprising ibudilast and bumetanide as active ingredients. In another preferred embodiment, the treatment comprises concomitant administration of a dosage form comprising ibudilast and a dosage form comprising bumetanide.

According to the invention, the two active ingredients, ibudilast and bumetanide, can be provided either in a single pharmaceutical composition or as a kit comprising a separate dosage form for each active ingredient.

A kit is herein defined as combination product provided as a package and containing several individual dosage forms that show a complementary effect when applied together. In this aspect, the effect achieved by a kit and a pharmaceutical composition are similar.

The pharmaceutical composition according to the invention has the advantage that it reduces the number of dosage forms that have be to be administered to a patient, thereby increasing patient compliance. Furthermore, providing the active ingredients as a fixed dosage form ensures that the correct dosages of each active ingredient are administered together.

In the kit according to the invention, the amount of each active ingredient can be adjusted individually, according to the specific requirements of different age groups or in alignment with personalized treatment plans.

As used herein, the term autism spectrum disorder (ASD) is understood to cover a family of neurodevelopmental disorders characterized by deficits in social communication and interaction and restricted, repetitive patterns of behavior, interests or activities. In the following, the terms “autism spectrum disorder”, “autism” and “ASD” are used interchangeably.

As used herein, the term “patient” refers to an “ASD patient” and is intended to cover not only humans diagnosed as having ASD, but also humans suspected of having ASD.

The person skilled in the art is well aware of how a patient may be diagnosed with ASD.

For example, the skilled person may follow the criteria set up in “American Psychiatric Association; Diagnostic and Statistical Manual of Mental Disorders (DSM-5) Fifth edition” to give a subject a diagnosis of ASD. Likewise, ASD patients may have been diagnosed according to standardized assessments tools including, ADOS, ADI-R, CARS, DISCO or M-CHAT.

In other cases, patients may have a well-established DSM-IV diagnosis of autistic disorder, Asperger's disorder, or pervasive developmental disorder not otherwise specified (PDD-NOS).

Additionally, the present invention may be useful for subjects fulfilling one or more of the following criteria: persistent deficits in social communication and social interaction across multiple contexts as manifested by the following, currently or by history; restricted, repetitive patterns of behavior, interests, or activities, as manifested by at least two of the following, currently or by history; symptoms present in the early development period (but may not become fully manifest until social demands exceed limited capacities, or maybe masked by learned strategies in later life); symptoms cause clinically significant impairment in social, occupational, or other important areas of current functioning; these disturbances are not better explained by intellectual disability (intellectual development disorder) or global development delay.

ASD may occur with or without accompanying intellectual and/or language impairment. It may be associated with a known medical or genetic condition or an environmental factor or other neurodevelopmental, mental, or behavioral disorders.

ASD may occur in different severity levels which may be classified according to impairment in social communication and in terms of restricted, repetitive behavior. The present invention may be applied to patients suffering from any severity level of ASD.

In one embodiment, the pharmaceutical composition or kit according to the present invention is for use in the treatment of ASD in a subgroup of ASD patients called ASD phenotype 1 patients.

As used herein, the terms “ASD phenotype 1” and “phenotype 1” are used interchangeably.

In one embodiment, ASD phenotype 1 patients are diagnosed as phenotype 1 patients by a diagnosis of ASD, and presence of the following clinical signs and symptoms:
an enlarged head size at any time point after birth and before 24th months of age, defined as a head circumference ≥75 pct percentile in the CDC growth chart,
a systematic aggravation of ASD behavioral symptoms during episodes of immune challenges, e.g., fever, infectious events and acute inflammation, and/or
a negative reaction to a challenge test with an Nrf2-activator such as sulforaphane.

Presence or absences of these clinical signs and symptoms may be determined with the help of standardized questionnaires to be answered by the patient or a caretaker. In a preferred embodiment, the ASD phenotype 1 patient shows all clinical signs and symptoms.

ASD phenotype 1 patients may be identified with the help of a challenge test as described in WO 2019086722 A1. Briefly, the concept of a challenge test is based on administration of an Nrf2-activator to an ASD patient. In ASD phenotype 1 patients, who already show an upregulation of the respective pathways, further activation of Nrf2 will lead to a worsening of core symptoms. Consequently, ASD phenotype 1 patients may be identified by a negative behavioral response to a challenge test.

The pharmaceutical composition and kit according to the invention comprise ibudilast. Ibudilast is an anti-inflammatory and neuroprotective oral agent, metabolized mainly by the liver, having the following chemical structure of Formula I

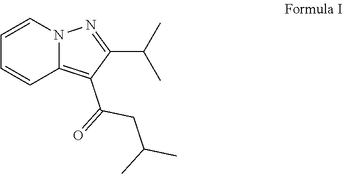 [SMILES: CC(C)CC(=O)c1c(C(C)C)nn2ccccc12]

Ibudilast is a phosphodiesterase (PDE) inhibitor, inhibiting mostly PDE4. The clinical efficacy of ibudilast has been proven for bronchial asthma and cerebrovascular disorders. Ibudilast is currently under clinical trial in the U.S. for progressive multiple sclerosis and other conditions such as amyotrophic lateral sclerosis and substances dependence (codes: AV-411 or MN-166).

The pharmaceutical composition or the dosage form of the kit according to the invention comprise between 2.5 mg and 50 mg ibudilast and are administered twice daily, so that the total daily dosage of ibudilast in the treatment according to the invention is between 5 and 100 mg ibudilast. In preferred embodiments, the pharmaceutical composition or the dosage form of the kit comprise 5 or 10 mg ibudilast, so that ibudilast is preferably administered at a total daily dosage of 10 or 20 mg.

The pharmaceutical composition and kit according to the invention further comprise bumetanide, also referred to as 3-(butylamino)-4-phenoxy-5-sulfamoylbenzoic acid. Bumetanide is an NKCC1 inhibitor and acts a loop diuretic. It is available under the tradenames bumex and burinex, among others. Its chemical structure is depicted below as Formula II

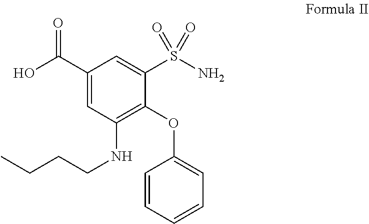 [SMILES: CCCCNc1cc(C(=O)O)cc(S(N)(=O)=O)c1Oc1ccccc1]

The pharmaceutical composition or the dosage form of the kit according to the invention comprise between 0.25 mg and 5 mg bumetanide and are administered twice daily, so that the total daily dosage of bumetanide in the treatment according to the invention is between 0.5 and 10 mg bumetanide. In a preferred embodiment, the pharmaceutical composition or the dosage form of the kit comprise 1 mg bumetanide, so that bumetanide is preferably administered at a total daily dosage of 2 mg.

In some embodiments, instead of bumetanide itself, the pharmaceutical composition or the dosage form of the kit according to the invention comprise derivatives of bumetanide, such as AqB007, AqB011, PF-2178, BUM13, BUM5 or bumepamine.

The pharmaceutical composition and kits according to the invention induce a favorable change in the electroencephalogram (EEG) of the patient.

An EEG is a recording of electrical activity in the brain of a subject, recorded, e.g., during rest or following stimulation. The observed signals can be grouped according to frequency and divided into alpha (8-13 Hz), beta (13-30 Hz), gamma (25-140 Hz), delta (0.5-4 Hz) and theta (4-7 Hz) waves. A healthy human brain shows certain patterns of activity in the EEG that correlate with the wakefulness of the person as well as the activities that the person is engaged in. Comparing the intensity and kind of activity in certain brain areas allows to determine normal and anormal brain function in a patient. Even though an anormal pattern may not necessarily result in symptoms, in ASD, subclinical electroencephalographic abnormalities (SEA) may still be associated with changes in intellectual functioning, dysfunctional behavior and severity of the disease.

Favorable changes in the EEG are therefore defined as any change in the EEG that make the EEG pattern in a certain brain area or during a certain activity more similar to that observed in an age- and sex-matched typically developing control.

Different parts of the brain are associated with different functionalities. For example, one or several brain areas are associated with cognition, including thinking, reasoning, problem-solving, memory, language; sensory processing such as hearing, sight, taste, smell or touch; motor control; memory, including short-term (working memory) and long-term (procedural memory); sleep and wakefulness; language processing, including comprehension and communication; learning and plasticity; emotions; social and behavioral functions.

Executive functions are higher-order cognitive processes that enable individuals to plan, organize, set goals, shift between tasks, and monitor their own behavior. Executive functions are primarily controlled by the prefrontal and frontal cortex, but also by the additional cortex including frontal anterior cingulate, lateral parietal cortex as well as the basal ganglia and the hippocampus.

Understanding language is controlled by various brain areas, including the Wernicke's area, located in the left superior temporal region; and the Angular gyrus located in the parietal lobe. Understanding language is also part of language processing which involves a larger network including temporal, frontal and parietal lobes.

Memory acquisition and memory processing involve multiple brain areas working together to encode, consolidate, store, and retrieve memories. These brain areas include hippocampus, cerebellum, temporal and superior temporal cortex, and parietal cortex.

In one embodiment, the brain area associated with executive function is the left and/or right frontal pole. In one embodiment, the brain area associated with understanding language and/or memory processing is the superior temporal region. In one embodiment, the brain area associated with memory processing is the entorhinal cortex. In one embodiment, the brain area associated with working memory function is the left superior area and/or the isthmus cingulate. In one embodiment, the brain area associated with sensory function is the paracentral region.

In a preferred embodiment, the favorable change in the EEG comprises a reduction of the gamma waves in the brain areas associated with executive function, understanding language and memory processing, i.e., the left and/or right frontal pole, the superior temporal region and/or the entorhinal cortex. In a particularly preferred embodiment, the gamma waves in the respective brain areas are reduced by 30 to 50% expressed in relative power. Therefore, in one embodiment, the favorable change in the EEG comprises a reduction of the gamma waves by 30 to 50% expressed in relative power in the left and/or right frontal pole. In one embodiment, the favorable change in the EEG comprises a reduction of the gamma waves by 30 to 50% expressed in relative power in the superior temporal region. In one embodiment, the favorable change in the EEG comprises a reduction of the gamma waves by 30 to 50% expressed in relative power in the entorhinal cortex. In a particularly preferred embodiment, the favorable change in the EEG comprises a reduction of the gamma waves by 30 to 50% expressed in the left and right frontal pole, the superior temporal region, and the entorhinal cortex.

Elevated gamma waves have been associated with altered sensory processing of stimulus characteristics and are suspected to primarily reflect local abnormal neuronal interaction caused by the imbalance of the excitatory interaction onto and the inhibition originating from parvalbumin positive interneurons (inhibitory neurons). The persistent excitatory GABA activity suggests that the ability to synchronize gamma power may be specifically reduced in ASD patients, as previously suggested for Fragile X syndrome patients. Such pattern of increased total high-frequency (gamma waves) neural activity but reduced temporally synchronous and spatially focused neural activity may have broad neurobehavioral implications in addition to its impact on sensory processing. As gamma frequency is the primary working frequency range in the human auditory system, it is possible to assess the desynchronization or synchronization of neuronal response to oscillating frequency of sensory stimulus. Using a chirp stimulus that increase linearly in frequency from 1 to 100 Hz has been used successfully to examine neuronal activity in the gamma frequency.

In another embodiment, the favorable change in the EEG comprises an increase of alpha waves in brain areas associated with working memory function and sensory functions, i.e., in the left superior area, the isthmus cingulate and/or the paracentral region. In a particularly preferred embodiment, the alpha waves in the respective brain areas are increased by 20 to 40% expressed in relative power. Therefore, in one embodiment, the favorable change in the EEG comprises an increase of the alpha waves by 20 to 40% expressed in relative power in the left superior area. In one embodiment, the favorable change in the EEG comprises an increase of the alpha waves by 20 to 40% expressed in relative power in the isthmus cingulate. In one embodiment, the favorable change in the EEG comprises an increase of the alpha waves by 20 to 40% expressed in relative power in the paracentral region. In a particularly preferred embodiment, the favorable change in the EEG comprises an increase of the alpha waves by 20 to 40% expressed in relative power in the left superior area, the isthmus cingulate and the paracentral region.

In another preferred embodiment, the favorable change in the EEG comprise both a reduction of the gamma waves by 30 to 50% expressed in relative power in the left and right frontal pole, the superior temporal region and the entorhinal cortex and an increase of the alpha waves by 20 to 40% expressed in relative power in the left superior area, the isthmus cingulate and the paracentral region.

In one embodiment, the treatment with the composition or kit according to the invention results in increased auditory sensory processing through neural auditory synchronization (or increased phase-locking) to an external auditory or chirp stimulus, wherein the increase is compared to the gamma band phase-locking prior to treatment. One way of measuring the increased auditory sensory processing is the following: patients hear an auditory chirp stimulus generated using a 1000-Hz tone that is amplitude modulated by a sinusoid linearly increasing in frequency from 1 to 100 Hz over 2 s. Auditory cortex signals are detected using sensors distributed across the fronto-central scalp. Analysis of time-frequency, single trial power (STP) and inter-trial coherence (ITC) is then achieved applying Morlet wavelets. Increase in ITC indicates increased neural synchronization to the auditory chirp.

Without wanting to be bound by a mechanism, the inventors believe that it is the reduction of the gamma waves in the fronto-central area that leads to increased auditory sensory processing. Therefore, in one embodiment, the invention relates to a pharmaceutical composition comprising ibudilast and bumetanide as active ingredients or a kit comprising a dosage form comprising ibudilast and a dosage form comprising bumetanide for use in the treatment of autism spectrum disorder (ASD) for use in the treatment of ASD, wherein the treatment induces a favorable change in the EEG, wherein the favorable change comprises a reduction of gamma waves in brain areas associated with executive function, understanding language and memory processing by 30 to 50% expressed in relative power and the treatment results in increased auditory sensory processing through neural auditory synchronization to an external auditory stimulus.

The pharmaceutical compositions and kits according to the invention are the first drug interventions available for the treatment of ASD that lead not only to an improvement of symptoms, but also induce quantifiable changes in the EEG.

The kits according to the invention are for use in a treatment of ASD, wherein the two dosage forms are administered concomitantly. The pharmaceutical composition and the dosage forms are administered twice daily (bid).

The pharmaceutical compositions and the dosage forms of the kit of the invention are preferably oral dosage forms, e.g., tablets, capsules, pills, sachets, powders, drops, emulsions, gels, gums, liquids, lyophilisates, pastes, solutions or suspensions, most preferably tablets, capsules or pills.

It is also envisaged by the present invention that the pharmaceutical composition or the dosage forms of the kit according to the present invention additionally comprise pharmaceutically acceptable carriers or excipients such as lubricants, disintegrants, adhesives, antiadhesives, binders, preservatives, fillers, sorbents, or vehicles.

The pharmaceutical composition and dosages forms of the invention may be formulated as immediate-release formulations or sustained-released/modified-release formulations. In preferred embodiments, the dosage form comprising ibudilast is a modified-release formulation and the dosage form comprising bumetanide is an immediate-release formulation. In one embodiment, the active ingredients are formulated differently in the pharmaceutical composition according to the invention, i.e., ibudilast is formulated for modified release and bumetanide is formulated for immediate release.

### Example

Effect of 2 Weeks Treatment with STP1 in ASD-Phen1 Patients

Patients were eligible to participate in the study if they met all of the following inclusion criteria: 1) Male or female individuals, aged between 18 and 40 years inclusive, previously diagnosed with ASD (based on Diagnostic and Statistical Manual of Mental Disorders, 5th Edition [DSM-5] criteria confirmed by a history and interview with an expert in autism diagnosis (CE); 2) a well-documented enlarged head size at any time point after birth and before 24 months of age, defined as a head circumference ≥75th percentile CDC growth charts (12) or diagnosed with macrocephaly; 3) a systematic aggravation of ASD behavioral symptoms, so-called flares occurring during episodes of immune challenges (such as fever and infectious events e.g., acute inflammation) as assessed by the ASD-Phen1 semi-structured interview form.

A total of 12 patients (median age 19.2 years) were screened for study eligibility, of which, 12 (100.0%) were randomized to treatment with STP1 5/1 mg (i.e., ibudilast 5 mg-bumetanide 1 mg; n=6) or placebo (n=2) in Cohort 1 and STP1 10/1 mg (i.e., ibudilast 10 mg-bumetanide 1 mg; n=3) or placebo (n=1) in Cohort 2.

Cohort 1 received SPT1-5/1 mg per os b.i.d. (5 mg ibudilast and 1 mg bumetanide, n=6) or placebo (n=2). Cohort 2 received STP1-10/1 mg per os b.i.d. (10 mg ibudilast and 1 mg bumetanide, n=3), or matching oral placebo twice daily (n=1).

The two drugs (ibudilast and bumetanide) were taken at the same time with food and water, and the two daily doses were separated by approximately six hours.

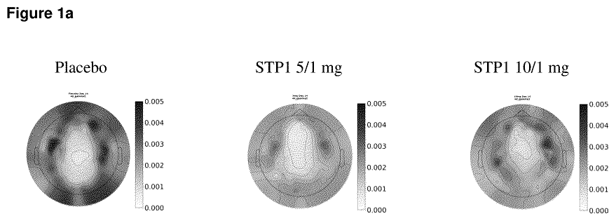

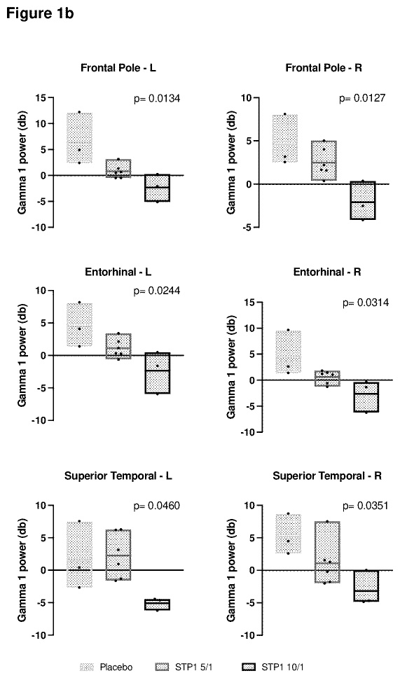

In brain regions of interest associated with executive function (frontal left and right poles) and memory (left and right entorhinal regions), STP1 induced a statistically significant dose-related effect in reducing gamma power in comparison to placebo (FIG. 1a and FIG. 1b). In particular, a significant effect was observed in brain regions of interest associated with social cognition and emotional processing for the left and right frontal poles (executive function), left and right entorhinal regions (memory-related processes), and left and right superior temporal region.

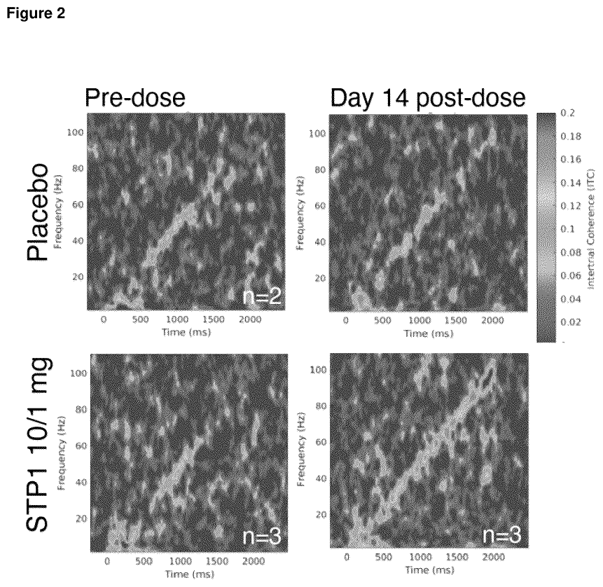

For the Chirp Analysis, 10 of 12 subjects with complete datasets were considered. Two subjects did not complete baseline chirp EEG. Responses include intertrial phase coherence (ITC; 40 Hz, 80 Hz, onset, and offset) and single trial power (STP; alpha, gamma1, and gamma2). Increased ITC indicates increased neural synchronization to the auditory chirp. STP refers to background spectral power during the auditory chirp. The ITC values are approximated from time-frequency analysis of the chirp response depicted in FIG. 2.

In the STP1 10/1 mg group (N=3), the provided mean score refers to change from baseline to either Day 1 (acute post-dose), Day 14, and Day 28. Mean score for follow-up Day 14 includes ITC 40 Hz (0.008, SD=0.086), ITC 80 Hz (0.030, SD=0.053), STP mean scores for follow-up Day 14 include alpha (−1.3, SD=6.8), gamma1 (−2.1, SD=2.7), gamma2 (−2.6, SD=1.7). Overall, there was a clear numerical increase in ITC 80 Hz neural synchronization as can be seen in FIG. 2.

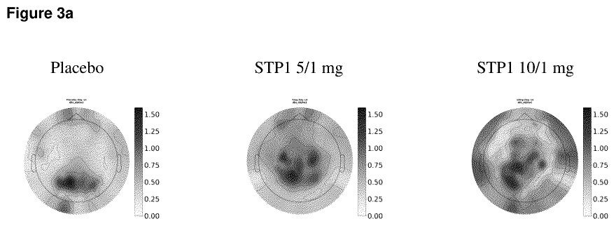

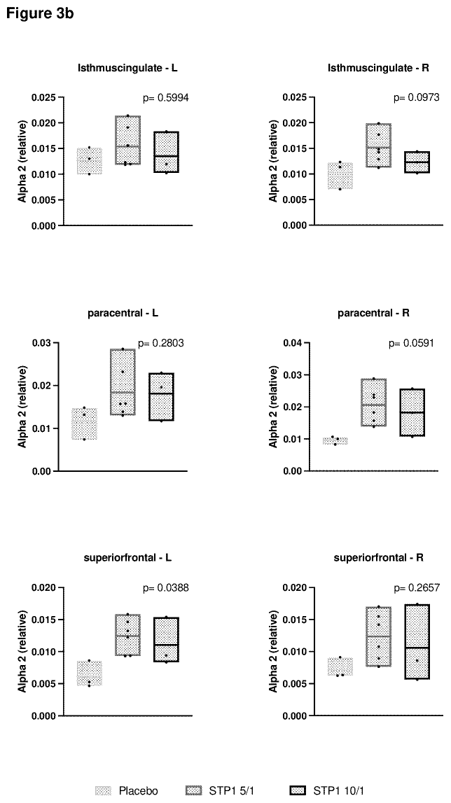

Treatment with STP1 showed a significant increase of alpha 2 power in frontal and occipital regions and in brain areas associated with memory function (frontal right poles, right isthmus cingulate) and sensory (right paracentral regions) (FIGS. 3a and 3b).

1. A method of treating autism spectrum disorder (ASD) comprising administering a composition comprising ibudilast and bumetanide as active ingredients, wherein the treatment induces at least one favorable change in the electroencephalogram (EEG) of the patient.

2. A kit comprising a dosage form comprising ibudilast and a dosage form comprising bumetanide for use in the treatment of autism spectrum disorder (ASD), wherein the treatment induces at least one favorable change in the electroencephalogram (EEG) of the patient.

3. The method of claim 1, wherein the at least one favorable change in the EEG comprises a reduction of gamma waves in brain areas associated with executive function, understanding language and memory processing.

4. The method of claim 3, wherein the brain area associated with executive function is the left and/or right frontal pole.

5. The method of claim 3, wherein the brain area associated with understanding language and memory processing is the superior temporal region.

6. The method of claim 3, wherein the brain area associated with memory processing is the entorhinal cortex.

7. The method of claim 3, wherein the gamma waves expressed in relative power in the respective brain areas are reduced by 30 to 50%.

8. The method of claim 1, wherein the at least one favorable change in the EEG comprises an increase of alpha 2 power in brain areas associated with working memory function and sensory functions.

9. The method of claim 8, wherein the brain area associated with working memory function is the left superior area and/or the isthmus cingulate.

10. The method of claim 8, wherein the brain area associated with sensory function is the paracentral region.

11. The method of claim 8, wherein alpha 2 waves expressed in relative power in the respective brain areas is increased by 20 to 40%.

12. The method of claim 1, wherein the treatment results in increased auditory sensory processing through neural auditory synchronization to an external auditory stimulus.

13. The method of claim 1, wherein the composition is administered twice per day.

14. The method of claim 1, wherein the composition is an oral dosage form.

15. The method of claim 1, wherein the treatment comprises administration of a total daily dose of ibudilast of between 5 mg and 30 mg and/or administration of a total daily dose of bumetanide of between 0.5 mg and 10 mg.

16. The method of claim 1, wherein the treatment comprises administration of a total daily dose of ibudilast of between 10 mg or 20 mg and/or administration of a total daily dose of bumetanide of 2 mg.

17. The method of claim 1, wherein the patient is an ASD phenotype 1 patient.

18. The method of claim 1, wherein the patient is diagnosed as an ASD phenotype 1 patient by a diagnosis of ASD, and
an enlarged head size at any time point after birth and before 24th months of age, defined as a head circumference ≥75 pct percentile in the CDC growth chart,
a systematic aggravation of ASD behavioral symptoms during episodes of immune challenges, e.g., fever, infectious events and acute inflammation, and/or
a negative reaction to a challenge test with an Nrf2 activator such as sulforaphane.
